(19) United States
(12) Patent Application Publication
(10) Pub. No.: US 2018/0036062 A1
(43) Pub. Date: Feb. 8, 2018

(54) DOUBLE-TIP MONOPOLAR ELECTROSURGERY DEVICE

(71) Applicant: Firas Eladoumikdachi, Princeton, NJ (US)

(72) Inventor: Firas Eladoumikdachi, Princeton, NJ (US)

(21) Appl. No.: 15/668,320

(22) Filed: Aug. 3, 2017

Related U.S. Application Data
(60) Provisional application No. 62/370,550, filed on Aug. 3, 2016.

(51) Int. Cl. A61B 18/14; A61B 90/00

(52) U.S. Cl. CPC A61B 18/14; A61B 90/39; A61B 2018/126

(57) ABSTRACT

The present invention provides a device for monopolar electrosurgery that comprises two parallel spaced active cutting electrodes. The two active electrodes provide simultaneous, parallel or concentric surgical cuts, one cut around the target tissue and the second cut defining the surgical tissue margin. The distance between the two active electrodes provides a relatively consistent tissue margin, so long as the relative orientation of the two active electrodes is maintained relative to the target tissue during the cut. In this way, a consistent margin is simultaneously provided at the same time that the target tissue is surgically removed.

### RELATED APPLICATIONS

This application claims the priority benefit of U.S. Provisional Application Ser. No. 62/370,550, filed Aug. 3, 2016.

### FIELD OF THE INVENTION

The invention relates to an electrosurgical device and particularly to a lumpectomy cutting device. More particularly, the invention relates to a double-tip monopolar electrosurgery device for lumpectomy procedures and similar procedures. The double-tip monopolar electrosurgery device of the invention dissects out target tissue (such as a tumor) while simultaneously excising a margin of tissue around the target tissue.

### BACKGROUND OF THE INVENTION

Lumpectomy is a surgical procedure that involves removing a suspected malignant (cancerous) tumor, or lump, and a small portion of the surrounding tissue (margin) from a woman's breast. The surgeon's goal during surgery is to take out all of the cancer along with a rim of normal tissue around it. During or after surgery, a pathologist examines this rim of tissue—called the surgical margin or margin of resection—to determine that it is clear of any cancer cells. If cancer cells are present, this will influence decisions about treatments such as additional surgery and radiation.

Typically, the surgeon utilizes a scalpel to cut around the target tissue and the rim of normal tissue. Conventional scalpels require multiple slicing strokes, which approaches carving and which can leave rough or jagged edges. While some current scalpels slice nicely, they still leave ‘train tracks’ (the narrow incisions) which make margin assessment inaccurate, and unnecessarily leave undesired cuts. Moreover, current techniques using multiple slicing movements around all sides of the tumor using available scalpels creates a challenge for properly orienting the excised specimen, thus causing inaccurate margin assessment.

As an alternative to conventional scalpels, electrosurgery (also referred to as electrocautery) may be used to cut around target tissue. In monopolar electrosurgery, tissue is cut and coagulated by completing an electrical circuit in which high frequency electric current from a generator is conducted through the surgical site with an active electrode. In monopolar electrosurgery, a single active electrode is used in the surgical site to function as the “blade” and cut the tissue. The electrosurgical current is then dispersed through the patient to a return electrode and returns to the generator to complete the path.

In bipolar electrosurgery, the current flows between two closely spaced bipolar electrodes that are positioned around tissue. Current passes from one electrode through the tissue to the other electrode, completing the electrical circuit without passing through any other part of the patient's body. Examples of bipolar devices are bipolar forceps. However, bipolar devices can't be used to cut through tissue.

Typically the target tissue (such as a tumor) and the margin are removed from the patient in two steps. Using current dissecting instruments, such as commercially available monopolar electrosurgery pens, a margin is excised only after the main tumor has been removed. Once the tumor is removed, the remaining tissue surrounding the surgical wound deforms. This can make it very difficult for the surgeon to accurately assess and remove a margin of tissue from around the wound that formerly contained the excised tumor. The present invention provides a means to address the above problems and provides for accurate margin resection.

### SUMMARY OF THE INVENTION

In at least one embodiment, the present invention provides a device for monopolar electrosurgery that comprises two parallel spaced active electrodes. The two active electrodes provide simultaneous, parallel or concentric surgical cuts, one cut around the target tissue and the second cut defining the outside of the margin. The distance between the active electrodes provides a consistent margin, so long as the relative orientation of the two active electrodes to the target tissue is maintained during the cut. In this way, a consistent margin is simultaneously provided at the same time that the target tissue is surgically removed.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a schematic representation of a double-tip monopolar electrosurgery device in accordance with an embodiment of the invention.

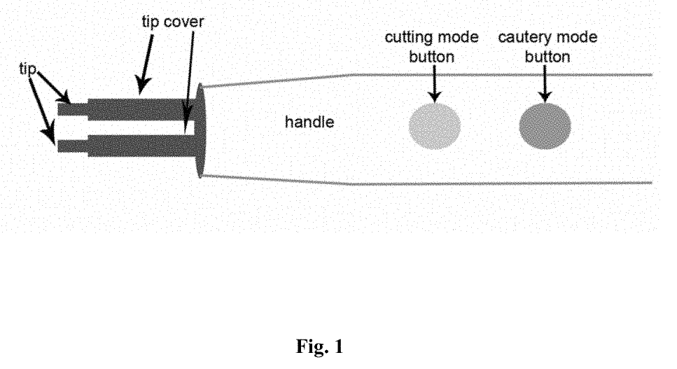

FIG. 2 provides a picture of a double-tip monopolar active electrode unit in accordance with an embodiment of the invention.

### DETAILED DESCRIPTION OF THE INVENTION

Certain terminology is used herein for convenience only and is not to be taken as a limitation on the present invention. The following describes preferred embodiments of the present invention. However, it should be understood, based on this disclosure, that the invention is not limited by the preferred embodiments described herein.

The present invention provides a monopolar device for electrosurgery. Accordingly, the active electrodes are placed in the surgical site and are used to cut tissue. A return electrode pad is attached elsewhere to the patient, and the high frequency electrical current then flows from a generator to the active electrodes, through the patient to the patient return electrode pad and back to the generator. The monopolar circuit thus includes a generator, active electrodes, patient, and return electrode pad.

In at least one embodiment, the present invention provides a device for monopolar electrosurgery that comprises two parallel, spaced active electrodes. The two parallel, spaced active electrodes are attached to a common handle such that while in use the two parallel, spaced active electrodes make simultaneous surgical cuts.

The invention also provides a double-tip active electrode unit. The double-tip active electrode unit comprises two parallel spaced active electrodes or electrode tips that are attached to a common base. As used herein, double-tip refers to monopolar devices having two active electrode cutting blades that can make simultaneous surgical cuts.

In use, one active electrode (the first active electrode) will provide the surgical cut proximal to the target tissue, such as a tumor. The device is oriented such that the second active electrode is distal from the target tissue, and during the surgical cut the second active electrode simultaneously provides a surgical cut that is parallel or concentric to the cut provided by the first active electrode. The distance between the first active electrode and the second active electrode provides a consistent margin, so long as the relative orientation of the two active electrodes is maintained during the cut. In this way, a consistent margin is simultaneously provided at the same time that the target tissue is surgically removed.

In at least one embodiment of the invention, the two active electrodes are interconnected to one another, either directly or by a handle assembly.

The two active electrodes are spaced apart at the appropriate distance to provide a tissue margin of the desired width. In some embodiments, the distance between the two monopolar active electrodes is between about 5 mm to about 12 mm, and more preferably from between about 5 mm to about 7 mm. In an embodiment of the invention, the distance between the two active electrodes is adjustable. In other embodiments, the distance between the two active electrodes is fixed.

In certain embodiments, the active electrode “blades” are flattened in shape. In preferred embodiments of the invention, each of the active electrodes has rounded blade edges, which prevent RF current from concentrating too much energy in one area of the tissue. The active electrodes may be constructed of stainless stain or other appropriate material. In certain embodiments the active electrodes are coated with non-stick PTFE.

In certain embodiments, the active electrodes are provided as sterile, disposable units for single use.

In certain embodiments, the target tissue may be a tumor. In specific embodiments, the target tissue may be described herein with respect to a lumpectomy of breast tissue. In other embodiments, the double-tip monopolar electrosurgery device may be designed and utilized for other procedures on tissue having similar texture, density, and physical characteristics for which the simultaneous excision of a margin is desired.

An electrosurgical generator is the source of the electric current used in the electrosurgery. Suitable monopolar electrosurgical generators are known in the art and are commercially available.

Because the devices of the present invention are monopolar, they are used with a return electrode that is affixed to the patient's body. The return electrode recovers the current from the patient's body and may be a conductive plate or pad. Suitable return electrodes are known in the art and are commercially available.

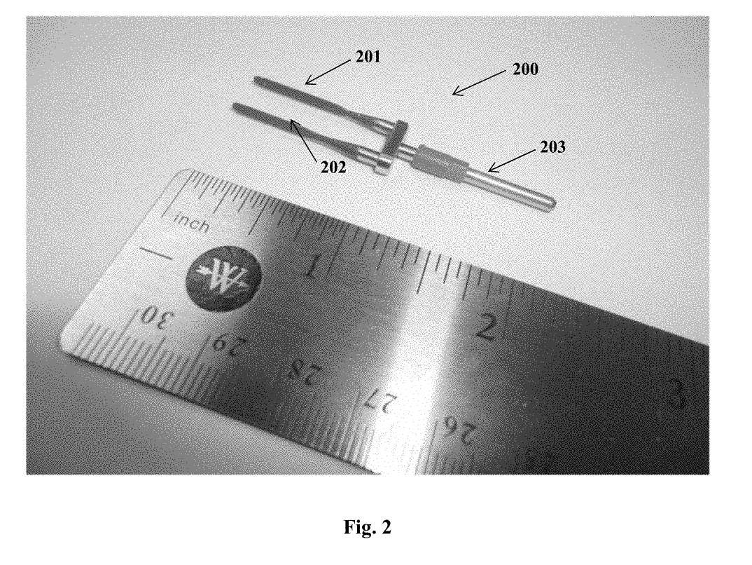

Referring to FIG. 2, the figure shows a double-tip active electrode unit 200. The electrode unit has two parallel, spaced active cutting electrodes 201, 202 (“blades”). As pictured, the two parallel active electrodes 201, 202 are flattened with rounded blade ends. In the illustrated embodiment, each of the blades is coated with a non-stick coating such as PTFE. The parallel spaced active cutting electrodes 201, 202 are attached to a shaft 203. The shaft 203 may be a standard size that allows for the use of the double-tip active electrode unit 200 with commercially available monopolar electrosurgery pens.

The monopolar electrosurgical double-tip device is positioned relative to the target tissue with one active electrode (i.e. 201) proximal to the target tissue and the other active electrode distal to the target tissue. The two active electrodes are engaged in a cutting mode and simultaneous cuts from the two active electrodes 201, 202 are provided with a single cutting action. The relative orientation of the two active electrodes relative to the target tissue is maintained to provide a consistent margin and standard excision (lumpectomy) is carried out. In this way, a consistent margin is simultaneously provided at the same time that the target tissue is surgically removed.

The described simultaneous removal of the target tissue and the margin tissue is believed to provide a more precise, less-jagged, smoother excision of tumors while at the same time providing a more precise definition of the surrounding resection of marginal tissue. It should be especially useful in obtaining a precise and accurate margin, and for providing better margin pathological classification (R0, R1, or R2) for verification of removal of all malignant tissue.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A device for monopolar electrosurgery that comprises:
two parallel spaced active electrodes attached to a common handle;
wherein the two parallel active electrodes make simultaneous surgical cuts.

2. The device for monopolar electrosurgery of claim 1, wherein the distance between the two parallel active electrodes is from about 5 mm to about 12 mm.

3. The device for monopolar electrosurgery of claim 2, wherein the distance between the two parallel active electrodes is from about 5 mm to about 7 mm.

4. The device for monopolar electrosurgery of claim 1, wherein each of the two active electrodes has a flattened shape.

5. The device for monopolar electrosurgery of claim 4, wherein each of the two active electrodes has rounded ends.

6. The device for monopolar electrosurgery of claim 5, wherein each of the two active electrodes is constructed of stainless stain and coated with non-stick PTFE.

7. A disposable electrode unit for monopolar electrosurgery comprising:
two parallel spaced active electrodes attached to a common base, wherein the two parallel active electrodes make simultaneous surgical cuts;
wherein the disposable electrode unit is provided as a sterile, disposable unit for single use.

8. The disposable electrode unit for monopolar electrosurgery of claim 7, wherein the distance between the two parallel active electrodes is from about 5 mm to about 12 mm.

9. The disposable electrode unit for monopolar electrosurgery of claim 8, wherein the distance between the two parallel active electrodes is from about 5 mm to about 7 mm.

10. The disposable electrode unit for monopolar electrosurgery of claim 1, wherein each of the two active electrodes has a flattened shape.

11. The disposable electrode unit for monopolar electrosurgery of claim 10, wherein each of the two active electrodes has rounded ends.

12. The disposable electrode unit for monopolar electrosurgery of claim 11, wherein each of the two active electrodes is constructed of stainless stain and coated with non-stick PTFE.
